(19) United States
(12) Patent Application Publication
(10) Pub. No.: US 2025/0212972 A1
(43) Pub. Date: Jul. 3, 2025

(54) GARMENT

(71) Applicant: WUKA LTD, Hemel Hempstead Hertfordshire (GB)

(72) Inventors: Rubina RAUT, Tewin (GB); David SLOCOMBE, Tewin (GB)

(21) Appl. No.: 18/852,348

(22) PCT Filed: Mar. 28, 2023
(86) PCT No.: PCT/EP2023/058066

§ 371 Date: Sep. 27, 2024

(30) Foreign Application Priority Data
Mar. 29, 2022 (GB) 2204471.3

(51) Int. Cl. A41B 9/00; A61F 13/49; A61F 13/496

(52) U.S. Cl. CPC A41B 9/001; A61F 13/49006; A61F 13/496

(57) ABSTRACT

A reusable menstrual and/or incontinence garment has a washable support sheet comprising a front portion, a rear portion and a crotch area, a gusset comprising a washable and reusable absorbent layer configured for the absorption of menstrual fluid and/or urine, and a first strap joining the front portion to the rear portion on a first side and a second strap joining the front portion to the rear portion on a second side, wherein the lengths of the first and second straps are adjustable.

### TECHNICAL FIELD

This invention relates to a reusable menstrual and/or incontinence garment. In particular, the invention relates to an adjustable menstrual and/or incontinence garment.

### BACKGROUND

Period underwear is absorbent clothing designed to be worn during menstruation. The underwear is washable and reusable, providing an environmentally friendly alternative to conventional disposable menstruation hygiene management products, such as menstrual pads and tampons. Likewise, incontinence underwear is a reusable undergarment designed to absorb urine. It provides an environmentally friendly alternative to traditional disposable incontinence products.

Conventional menstruation hygiene management products, such as menstrual pads and tampons are sold in a single size, with different absorbencies. The user buying the menstrual pad or tampon will not be concerned with whether the product will fit, solely whether the product has the correct absorbency. This simplicity of one-size products provides ease of distribution to locations that are only suitable for “universal” product items, such as vending machines.

Period underwear may be provided in eight or more sizes and three or more absorbencies, which would require at least 21 different items to be held in stock. This is a barrier to stocking period underwear as it requires a large amount of retail space, a high cost of maintenance of stock levels and has an impact on cash flow locked up in stock. Thus, conventional period underwear is unsuitable for locations such as vending machines, airports, hotels, and convenience shops. Locations where menstrual and/or incontinence products are needed often stock disposable products such as tampons or pads due to these problems.

### SUMMARY OF THE INVENTION

An aspect of the present invention provides a reusable menstrual and/or incontinence garment comprising:
a washable support sheet comprising a front portion, a rear portion and a crotch area;
a gusset comprising a washable and reusable absorbent layer configured for the absorption of menstrual fluid and/or urine; and
a first strap joining the front portion to the rear portion on a first side and a second strap joining the front portion to the rear portion on a second side, wherein the lengths of the first and second straps are adjustable.

The adjustable nature of the menstrual and/or incontinence garment enables a single sized garment to fit multiple dress sizes, whilst still ensuring a good fit. A further benefit is that if the wearer is between sizes or experiences bloating during their period the underwear can be made to fit their body rather than the body fitting a predefined size.

Suitable fabrics for the support sheet include cotton, rayon, viscose, modal, nylon, elastane. Recycled fabrics, such as recycled nylon may be used. The support sheet may comprise elastance or other suitable fabrics to give stretch. For example, the support sheet may comprise 5-10% elastance; optionally the support sheet may comprise 7-8% elastane.

The first and second straps may be configured to have an unstretched minimum distance between front and rear portions, and wherein the lengths of the first and second straps can be expanded without stretching by an additional 50-100% of the minimum distance.

The minimum distance may be between about 7 cm to about 14 cm. The minimum distance may be between about 12 cm to about 23 cm.

The first and second straps may be further expended by the stretch of the strap fabric, for example by at least about 40%, at least about 50%, at least about 60%, at least about 70%.

The first and/or second straps may have a width of from about 1 cm to about 2 cm. The first and/or second straps may have a thickness of from about 0.5 mm to about 3 mm.

The first and second straps may comprise an elasticated fabric. The elasticated fabric may be selected from cotton, modal (such as Tencel™), nylon or similar fabrics in natural fibre or blended with elastane for increased stretch.

The first and second straps may be detachably connected to the front and rear portions. This has the advantage of enabling the straps to be removed for washing and also enabling the garment to be removed/replaced without the need to undress.

The first and/or second straps may comprise an adjustable fastening means, allowing adjustment of the length of the first and/or second straps and fastening at the desired length.

The adjustable fastening means may comprise any suitable fastening, for example a hook and loop fastener (such as Velcro™), buckle fastening, clasp fastening, poppers etc.

The adjustable fastening means may comprise a sliding means, comprising a frame and a central bar forming two loops within the frame. The sliding means may be configured such that an end of the strap is attached to the bar and portions of the strap pass through the two loops. The frame may have a substantially rectangular shape, with straight edges parallel to the bar. One or both of the straight edges may be provided with a textured surface, such as teeth, on a surface facing the bar.

The first and second straps may be connected to the washable support sheet via connections means. The connection means may comprise any suitable means for connecting the first and second straps to support sheet, including stitching, rivets, hook and loop fastening and sliders.

The connection means may be configured so that at least one of the first and second straps are detachable.

The connections means may comprise a first section for connection to the washable support sheet and a second section for connection to the first or second strap, wherein at least one of the first and second section are configured to enable disengagement of the strap from the washable support sheet.

At least one of the first and second sections may comprise a loop, the loop comprising an opening, through which fabric of the strap or support sheet can pass.

The loop may comprise a frame member defining a central elongate space, wherein the opening is located in the frame member, at a position off set from the longitudinal axis of the elongate space.

The second section may comprise a loop, and wherein the opening is configured to be smaller than the width of the strap but larger than the thickness of the strap.

The relationship between the size and shape of the underwear is important in ensuring that underwear is functional and comfortable. The applicants have discovered that the shape of the underwear can be configured so that the function and comfort is maintained over a range of sizes.

The washable support sheet may be configured to have a first end, a second end and opposite sides, wherein the first end is at the front portion and the second end is at the rear portion.

The first and second side straps may connect to the washable support at the sides of the washable support at locations adjacent the first and second ends.

The distance between opposite sides at the second end may be larger than the distance between opposite sides at the first end. This results in the rear portion being wider than the front portion in the region of the waist. This has been found to provide a better fit across a range of sizes.

The distance between opposite sides at the second end may be larger than the distance between opposite sides at the first end by about 1 to about 10 cm. In one embodiment, the width of the second end is larger than the width of the first end by about 3 cm to about 6 cm.

In one embodiment, the distance between opposite sides at the second end is in the range of from about 23 cm to about 26 cm, preferably about 24.5 cm, and the distance between opposite sides at the first end is in the range of from about 19 cm to about 22 cm, preferably about 20.5 cm.

In another embodiment, the distance between opposite sides at the second end is in the range of about 28 cm to about 32 cm, preferably about 30 cm, and the distance between opposite sides at the first end is in the range of from about 23 cm to about 27 cm, preferably about 25 cm.

The side edges at the rear portion may be curved, configured to curve outwards from a centre line of the support sheet.

This feature ensures a good fit across a range of sizes, reducing the risk of the rear portion bunching between the gluteal muscles. In addition, the curved shape enables a wider gusset to be used, providing enhanced absorption and retention of period flow.

The garment may have a front height defined by the distance from the top edge of the front portion to the bottom of the front portion when the edges of the front and rear portions are aligned, wherein the front height is in the range of about 26.5 to about 29.5. The dimensions of the front height have been selected to provide a suitable front height across a range of sizes.

The gusset may have a length in the range of from about 20% to about 100% of the length of the support sheet. The gusset may have a length of at least about 20%, at least about 30%, at least about 40%, at least about 50%, at least about 60%, at least about 70%, at least about 80%, or at least about 90% of the length of the support sheet. The length of the support sheet is the distance between opposite ends of the support sheet. The gusset may extend to the edge of the support sheet on at least one of the front and rear portions. The gusset may extend to the edge of the support sheet at both front and rear portions.

The gusset may have a minimum width in the range of from about 7 cm to about 9 cm. For some styles, such as a thong, a thinner width of gusset is required. For example, garment comprising a thong may comprise a garment having a minimum with of at least 3 cm.

This has the advantage of providing enhanced absorption and retention of fluids.

The gusset may comprise an inner layer, i.e. which in use lies next to the skin, an outer layer and middle layers between the inner and outer layers. Each of the inner, outer, and middle layers are washable and reusable. The inner layer may be configured to wick moisture away from the body. The outer layer may be water-impermeable to prevent fluid from leaking. The middle layers are super-absorbent layers which both absorb and retain fluid.

Suitable fabrics for the inner layer include woven or knitted textile materials, such as bamboo, cotton or polyester. Suitable fabrics for the middle layer include bamboo, cotton, merino wool or synthetic fibres. The middle layer may comprise felts, woven, knitted brushed textile materials and/or non-woven fabrics. The knitted brushed textile material may comprise one or more of cotton, polyester, viscose, and polypropylene.

Suitable fabrics for the liquid impermeable fabric include thermoplastic polyurethanes (TPU). The outer layer may comprise a non-woven fabric.

The support sheet may comprise a high stretch fabric. A high stretch fabric may stretch by at least 10%, optionally by at least 30%, or by at least 50%. The support sheet may have a 2-way stretch (i.e. stretch in one direction) or a 4-way strength (i.e. stretch in two directions). The support sheet may comprise one or more panels comprising a high stretch fabric. The one or more panels may be located at the first and/or second end of the support sheet. The high stretch fabric may be configured to enable stretch in the direction extending from gusset to first or second end. The high stretch fabric may comprise a pleated fabric. Examples of suitable high stretch fabrics include elastomeric fabrics, such as high elastane content nylon or polyester.

The garment may be configured to be a thong. In a thong, the rear portion is configured to fit between the gluteal muscles of the wearer. In this embodiment, the width of the rear portion is less than the width of the front portion. In one embodiment, the end of the rear portion is configured to have a width of less than 3 cm.

Throughout the description and claims of this specification, the words “comprise” and “contain” and variations of the words, for example “comprising” and “comprises”, mean “including but not limited to”, and do not exclude other components, integers or steps. Moreover, the singular encompasses the plural unless the context otherwise requires: in particular, where the indefinite article is used, the specification is to be understood as contemplating plurality as well as singularity, unless the context requires otherwise.

All dimensions refer to dimensions of a material in its unstretched state unless a stretched state is specified.

Preferred features of each aspect of the invention may be as described in connection with any of the other aspects. Within the scope of this application, it is expressly intended that the various aspects, embodiments, examples and alternatives set out in the preceding paragraphs, in the claims and/or in the following description and drawings, and in particular the individual features thereof, may be taken independently or in any combination. That is, all embodiments and/or features of any embodiment can be combined in any way and/or combination, unless such features are incompatible.

### BRIEF DESCRIPTION OF THE DRAWINGS

One or more embodiments of the invention will now be described, by way of example only, with reference to the accompanying drawings, in which:

FIGS. 1a and 1b are front views of an embodiment, at minimum and maximum adjustments respectively;

FIG. 2 is a plan view of the unassembled garment of FIGS. 1a and 1B;

FIGS. 3A and 3B are plan views of different embodiments of fastening means;

FIGS. 4A and 4B are schematic front views of symmetric and asymmetric embodiments respectively;

FIG. 5A is a front view showing the front portion only;

FIG. 5B is a back view showing the rear portion only;

FIGS. 6A and 6B are front views of an embodiment;

FIGS. 7A and 7B are front and rear views of an embodiment; and

FIGS. 8 is a front view of an embodiment.

### DETAILED DESCRIPTION

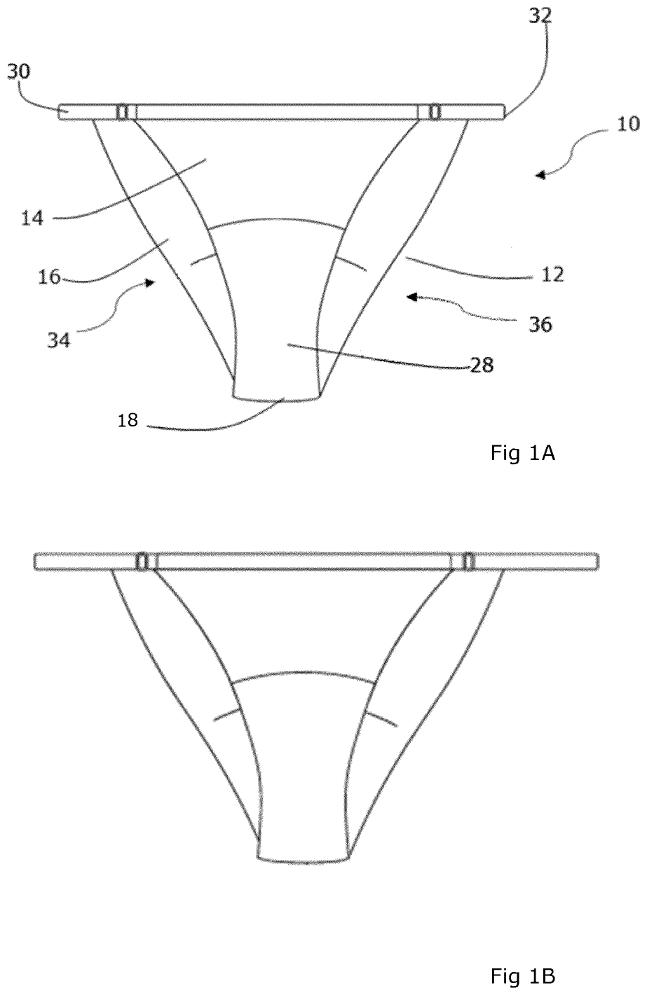

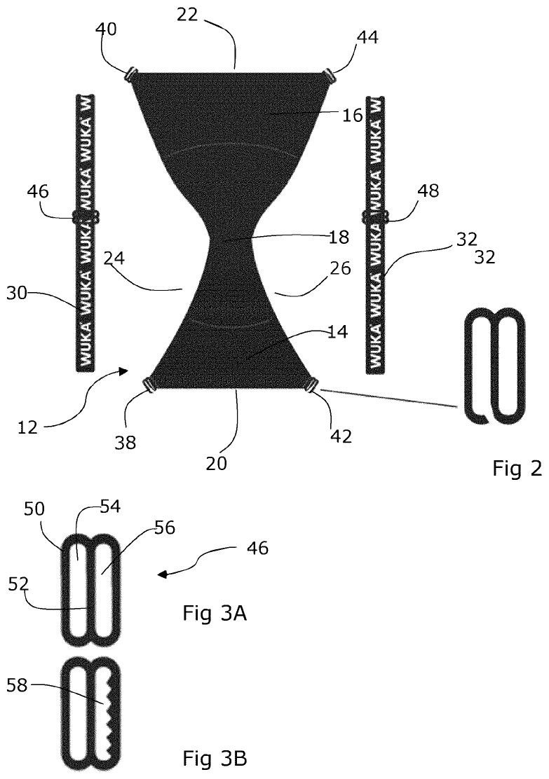

An underwear garment according to a first embodiment is illustrated in FIGS. 1a, 1b and 2.

The garment 10 has a washable support sheet 12 made up of a front portion 14, a rear portion 16 and a crotch area 18. The support sheet 12 has a first end 20, a second end 22 and opposite sides 24,26. The front portion 14 is defined between first end 20, the crotch area 18 and opposite sides 24,26. The rear portion 16 is defined between second end 22, the crotch area 18 and opposite sides 24,26.

A gusset 28 is provided in the crotch area 18, extending at least partially into one or both of the front and rear portions 14,16. The gusset 28 includes a washable and reusable absorbent layer which absorbs menstrual fluid and/or urine. The gusset will typically be formed from an inner layer, which in use lies adjacent the skin, one or more middle layers and an outer layer. The inner layer is configured to wick moisture from the skin towards the middle layer; the one or more middle layers are absorbent and are configured to absorb and retain menstrual fluid and/or urine. The outer layer is configured to be liquid impermeable and prevent menstrual fluid and/or urine from passing through.

A first strap 30 joins the front portion 14 to the rear portion 16 on a first side 24. A second strap 32 joins the front portion 14 to the rear portion on a second side 26. When assembled, a first leg hole 34 is defined by side 24 and first strap 30 and a second leg hole 36 is defined by side 26 and second strap 32.

As illustrated in FIG. 2, the first and second straps 30,32 are attached to the front and rear portions by connectors 38-40.

Each of first and second straps 30,32 are provided with an adjustable fastening means in the form of a sliding means, such as sliders 46,48. In use, the first strap 30 is attached at one end to connector 38, passes through connector 44 and is attached at its second end to slider 46. The strap also passes through portions of slider 46. This arrangement enables the slider to move along the strap, adjusting the amount of overlap of regions of the strap. This is a similar arrangement as found in bag handles and bra straps. A similar arrangement is used for the second strap 32. This allows the strap 30 to be adjusted to a minimum length, in which there is maximum amount of overlap of strap portions and maximum length, in which there is minimum amount of overlap of strap portions.

The arrangement of the garment allows it to be adjusted to fit a range of dress sizes. As shown in FIGS. 1A and 1B, side straps can be adjusted to adjust the waist size of the garment. Both FIGS. 1A and 1B depict the same garment, however FIG. 1A shows the side straps adjusted for minimum waist size and FIG. 1B shows the side straps adjusted for maximum waist size.

For both comfort and function, it is important that underwear has a good and secure fit across the range of dress sizes. The underwear has several features which optimise the fit.

FIGS. 3A and 3B show a slider 46 in more detail. The slider has a frame 50 and a central bar 52, the frame and central bar defining two loops 54,56. An end of a strap is attached to the bar 52, for example by folding it over the bar and sewing in place. The other end of the strap is threaded through a first connector, threaded through both loops 54,56 of the slider and then attached to the second connector, again by threading through, folding and sewing in place. In use, the slider 46 can be slid up and down the strap, thereby adjusting its length.

It has been found that a frame with straight external edges provides a firm grip on the strap, holding it firmly at the adjusted position. A straight edged frame ensures the flow of the fabric in the strap is directed horizontally, giving some reinforcement to the direction of fabric, whereas in a normal brief there would be much more fabric at the sides. By contrast, a circular frame would cause the underwear side straps to pull up over the hip and the underwear gusset to drop down. A round slider would allow the strap to rotate, and this would have the effect of enabling the strap to pull up and the underwear to slide down, making for either a very uncomfortable fit or underwear that can potentially fall down.

The hold of the slider can be improved by providing a textured surface on the inside surface of the sides of the frame, for example teeth 58 as illustrated in FIG. 3B.

The use of a strap to connect front and rear portions has the advantage that the resulting leg holes 34-36 are large enough to fit all sizes. This differs from standard underwear, in which the size of leg-hole is defined by the dress size, with the leg holes of smaller sizes being too small for larger sizes. The present invention therefore has the advantage that there is no sizing consideration for leg fitment, as one underwear garment of the present invention will fit all leg sizes.

Connectors 38-44 have a similar construction as the sliders, with a frame and central bar. FIG. 2 shows an enlarged image of connector 42, showing a similar arrangement as the slider of FIG. 3A. The connectors (and optionally the sliding means) may be configured to have an opening, thereby allowing the adjustable strap to be removed. This feature improves accessibility, enabling the underwear garment to be put on and removed without having to remove trousers, shoes or other clothing, as is possible when removing or applying a tampon or disposable pad. As illustrated in FIG. 2, the opening in the connector (or sliding means) is configured so that strap must be manipulated in two dimensions to move through the opening. For example, both upwards and sideways motion is required in the present embodiment. This prevents the strap from moving through the opening unintentionally during use.

In addition to an adjustable waist size provided by adjustable straps, it is important that the gusset is correctly and surely positioned across the range of dress sizes. The applicants have discovered that the shape of the garment can be optimised to ensure a good fit across a range of sizes.

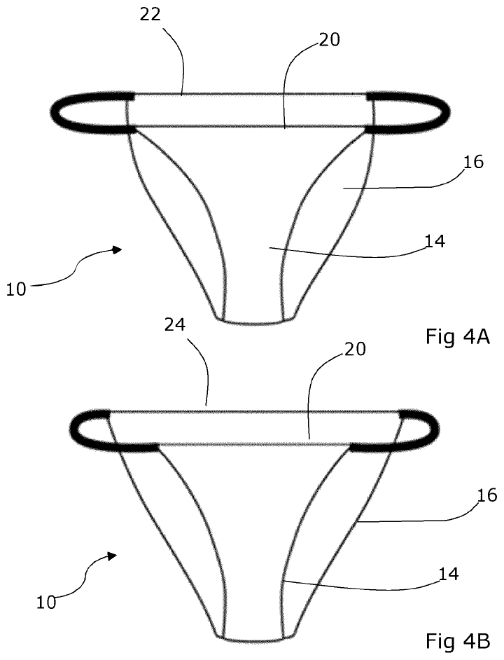

The applicants have discovered that the garment 10 provides a more secure fit across a range of dress sizes if the rear portion is wider than the front portion. FIG. 4A illustrates a garment in which the waist is symmetrical front 14 and back 16, i.e. the width of the first end 20 is equal to the width of the second end 22. This arrangement results in a relatively narrow rear portion, which can bunch between the gluteal muscles. FIG. 4B illustrates a garment in which the waist is asymmetrical front 14 and back 16. In this embodiment, the second end 22 is wider than the first end 20. This arrangement provides more rear coverage, resulting in the underwear remaining correctly in place. This correct positioning of the rear portion ensures that the gusset, including its absorbent layer, is held securely in position against the wearer's body. Furthermore, the resulting larger surface area of the rear portion enables a gusset with a larger absorbent layer to be used.

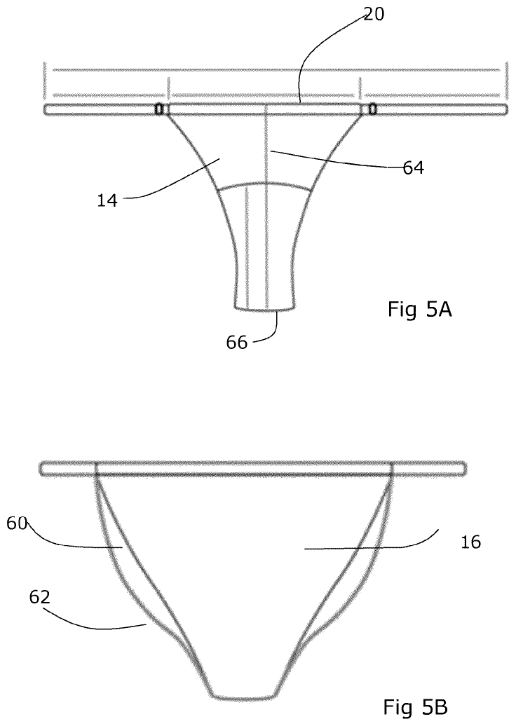

The applicants have discovered that the cut of the rear portion can be optimised to enhance the secure fitting of the underwear. FIGS. 5A and 5B show the front 14 and rear 16 of the underwear garment respectively. For clarity, FIG. 5A does not show the rear portion and FIG. 5B does not show the front portion. FIG. 5B shows two alternative shapes of the rear portion. The first shape of the rear portion has substantially straight sides 60, whereas the second shape has curved sides 62. The second shape provides a wider cut of the rear portion and a more secure fit across a range of sizes. This second shape has the additional advantage of enabling an increased surface area of the absorbent layers in the gusset. Thus, the curved shape of the rear portion enables maximum absorbency by enabling the surface area of the absorbent layers to be increased.

Different sized underwear not only has different side waists but also different sizes of front height. Front height is determined when the first and second ends 20,22 of the support sheet are placed together. In this position, the front height 64 is the distance between the first end 20 at the top of the underwear and the folded end 66 of the gusset at the bottom. The front height 64 must be selected so that it is appropriate for the full range of sizes accommodated by the adjustable underwear, so that the waists sits higher on a smaller body and lower on a larger body without becoming uncomfortable, loose or revealing for either. By contrast, fitting an expandable strap to conventional underwear would result in underwear that is not suitable for the full range of sizes. The combined dimensions ensure the underwear stays close to the body around all seams to prevent leakage.

As described above, the fit of the underwear on the vertical axis changes as the underwear size changes. Therefore, underwear which is a midi brief height on an XS wearer might become a hipster height underwear on a size L. This is because the L size wearer is generally taller than the XS wearer, although not always. The applicants have discovered that this problem can be overcome by the use of high stretch fabric and/or pleated fabric to allow the fabric to stretch on the vertical axis as well as expand using the side straps on the horizontal axis (waist).

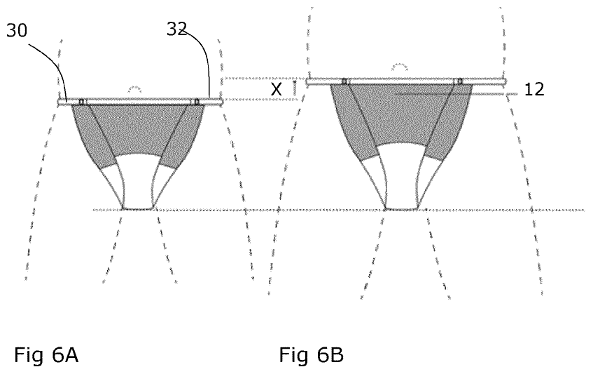

FIGS. 6A and 6B illustrate front views of an underwear garment in which the support sheet comprises a high-stretch fabric, such as elastane. FIG. 6A illustrates the underwear worn by a size XS wearer and FIG. 6B illustrates the underwear worn by a size L wearer. The adjustable straps enable a single underwear garment to fit both sizes around the waist. The high-stretch fabric of the support sheet enables the waist of the underwear to be positioned at the same position relative to the wearer's body in both sizes (in the embodiment of FIGS. 6A and 6B, this is just below the navel). The difference in front height between the underwear illustrated in FIG. 6A and 6B is shown as X. This difference is provided by the stretch in the support sheet 12. The underwear garment therefore provides a good fit for sizes XS, L and sizes in between. Whilst the underwear garment in FIGS. 6A-6B uses a high-stretch fabric for the support sheet, it is possible that only sections of the support sheet comprise high-stretch fabric, such as the sections which in use will be at the waist of the underwear garment.

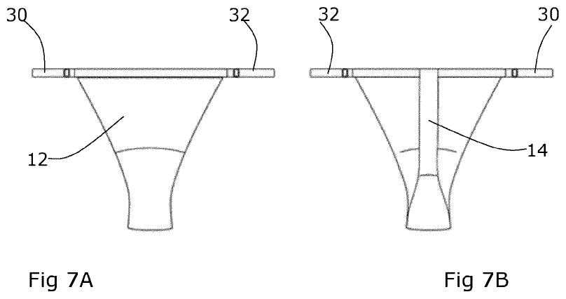

An embodiment in which the underwear garment is a thong is illustrated in FIGS. 7A and 7B. In this embodiment, the front portion 12 is similar to the embodiment of FIG. 1A and 1B. However, the back portion 14 is sufficiently narrow to fit between the gluteal muscles of the wearer. First and second straps 30,32 connect the front and back portions 12,14 as in previous embodiments.

Table 1 below provide some dimensions of an underwear garment which can be adjusted to accommodate sizes XS, S, M and L. The adjustable strap on each side of the underwear allows an increase from minimum strap length 18 cm (i.e. each strap is 9 cm) to maximum strap length 33 cm (i.e. each strap is 16.5 cm) which is an increase in 15 cm. A 15 cm expansion allows for a range of four sizes in universal sizing.

TABLE 1

| | Expansion | Front and | Total | |
|---|---|---|---|---|
| | strap | back width | stretched | |
| | length | of underwear | waist size | Universal |
| | (cm) | fabric | (cm) | size |
| Smallest expansion | 18 | 36 | 90 | XS |
| strap position | | | | |
| Maximum extended | 33 | 36 | 115 | L |
| expansion strap | | | | |
| position | | | | |

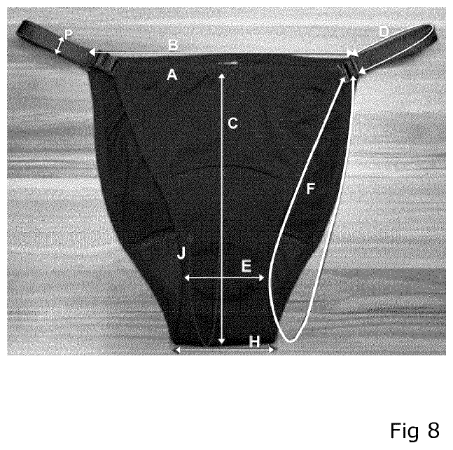

Table 2 gives the dimensions of two sizes of underwear garment. The first size is XS-L, covering XS, S, M, L and the second size is XL-4XL, covering XL, 2XL, 3XL and 4XL. Features A-P of the underwear garment are labelled on FIG. 8. Features A-P are measured with the garment laid flat on a surface with top of front portion (end 20) in line with the top of the rear portions (end 22). All measurements are taken with the fabric unstretched. Length of the absorbency layer J includes sections on both the front and back portions. To work effectively across the range of sizes, there is a relationship between features A-P.

TABLE 2

| | | XS-L | XL-4XL | Tolerance +/− |
|---|---|---|---|---|
| | Underwear Feature | (cm) | (cm) | (cm) |
| A | Front waist width | 20.5 | 25 | 1 |
| B | Back waist width | 24.5 | 30 | 1 |
| C | Mid rise | 26.5 | 29.5 | 1 |
| D | Side strap length (at longest | 17 | 19 | 0.8 |
| | adjustment) | | | |
| D | Side strap length (at shortest | 9.5 | 10.5 | 0.8 |
| | adjustment) | | | |
| E | Width of front gusset (at | 8 | 8.5 | 0.5 |
| | narrowest section) | | | |
| F | ½ leg opening | 25.2 | 28.8 | 1 |
| H | Width of crotch | 9.2 | 10 | 0.5 |
| J | Length of absorbency layer | 26.5 | 34 | 0.8 |
| P | Side strap width | 1.5 | 1.5 | |

Although Table 2 shows garments in sizes XS-L and XL-4XL, other size ranges may be chosen, for example XXS-M/L and L-3XL

These garments have the advantage that a single garment can be adjusted to fit several dress sizes. The design of the garment enables it to absorb and hold period flow and/or urine for these different body types without leakage from sides, front or back during use.

1. A reusable menstrual and/or incontinence garment comprising:
a washable support sheet comprising a front portion, a rear portion and a crotch area;
a gusset comprising a washable and reusable absorbent layer configured for the absorption of menstrual fluid and/or urine; and
a first strap joining the front portion to the rear portion on a first side and a second strap joining the front portion to the rear portion on a second side, wherein the lengths of the first and second straps are adjustable.

2. A garment according to claim 1, wherein the first and second straps are configured to have an unstretched minimum distance between front and rear portions, and wherein the lengths of the first and second straps can be expanded without stretching by an additional 50-100% of the minimum distance.

3. A garment according to claim 2, wherein the minimum distance is between about 7 cm to about 14 cm.

4. A garment according to claim 2, wherein the minimum distance is between about 12 cm to about 23 cm.

5. A garment according to any preceding claim, wherein one or both of the first and second straps are detachably connected to the front and/or rear portions.

6. A garment according to any preceding claims, wherein the first and/or second straps comprise an adjustable fastening means, allowing adjustment of the length of the first and/or second straps and fastening at the desired length.

7. A garment according to any preceding claim, wherein the adjustable fastening means comprises a sliding means, the sliding means comprising a frame and a central bar forming two loops within the frame.

8. A garment according to claim 7, wherein the frame has a substantially rectangular shape, with straight edges parallel to the bar.

9. A garment according to claim 8, wherein one or both of the straight edges is provided with textured surface on a surface facing the bar.

10. A garment according to any preceding claim, wherein the first and second straps are connected to the washable support sheet via connections means.

11. A garment according to claim 10, wherein the connection means are configured so that at least one of the first and second straps are detachable at one or both ends.

12. A garment according to claim 11, wherein the connection means comprises a first section for connection to the washable support sheet and a second section for connection to the first or second strap, wherein at least one of the first and second section are configured to enable disengagement of the strap from the washable support sheet.

13. A garment according to any preceding claim, wherein the gusset has a length in the range of from about 20% to about 100% of the length of the support sheet.

14. A garment according to any preceding claim, wherein the gusset extends to the edge of the support sheet on at least one of the front and rear portions.

15. A garment according to any preceding claim, wherein the support sheet comprises a high stretch fabric which stretches by at least 10%, optionally by at least 30%.

16. A garment according to claim 15, wherein the support sheet comprises a first panel of high stretch fabric at an end of the front portion and a second panel of high stretch fabric at an end of the rear portion.

17. A garment according to any preceding claim, wherein side edges at the rear portion are configured to curve outwards from a centre line of the support sheet.

18. A garment according to any preceding claim, wherein the washable support sheet is configured to have a first end, a second end and opposition sides, the first end being provided at the front portion and the second end being provided at the rear portion, wherein the distance between opposite sides at the second end is larger than the distance between opposite sides at the first end.

19. A garment according to claim 18, wherein the distance between opposite sides at the second end is larger than the distance between opposite sides at the first end by about 1 cm to about 10 cm when the garment is unstretched.

20. A garment according to any preceding claim, wherein the gusset has a minimum width in the range of from about 7 cm to about 9 cm.

21. A garment according to any of claims 1-16, in which the garment is configured to be a thong, wherein the rear portion is configured to fit between gluteal muscles of the wearer.

22. A garment according to claim 21, wherein the gusset has a width of at least 3 cm.

23. A garment according to any preceding claim, wherein the first and second side straps comprise a fabric which can stretch by at least 40%.
